(19) United States
(12) Patent Application Publication
(10) Pub. No.: US 2014/0236489 A1
(43) Pub. Date: Aug. 21, 2014

(54) BLOOD GLUCOSE MEASURING KIT CONNECTABLE TO MOBILE DEVICE

(71) Applicant: Chien-Chen CHEN, Taipei City (TW)

(72) Inventor: Chien-Chen CHEN, Taipei City (TW)

(21) Appl. No.: 13/772,339

(22) Filed: Feb. 21, 2013

(51) Int. Cl. G01N 33/49

(52) U.S. Cl. CPC G01N 33/49
USPC 702/19

(57) ABSTRACT

A blood glucose measuring kit connectable to a mobile device is configured to display blood glucose data via the screen of an external mobile device, with a view to reducing the production cost of the measuring kit. The measuring kit includes a housing provided therein with a control unit, a test strip receiver, and a data transfer port. The control unit generates blood glucose data by testing a blood glucose test strip with the test strip receiver and outputs a blood glucose test completion signal. The data transfer port is electrically connected to the mobile device and transfers the blood glucose data thereto. Using a signal processing program downloaded wirelessly from the Internet, the mobile device reads, analyzes, applies statistics to, and stores the blood glucose data, displays the blood glucose data on its screen, and can upload and download the blood glucose data wirelessly to and from the Internet.

### BACKGROUND OF THE INVENTION

1. Technical Field

The present invention relates to a blood glucose measuring kit connectable to a mobile device. More particularly, the present invention relates to a blood glucose measuring kit configured to display blood glucose data by means of the screen of an external mobile device so as to reduce the production cost of the measuring kit.

2. Description of Related Art

A blood glucose meter is a common example of physiological measurement devices and is used in the following manner. A blood glucose test strip on which a drop of blood has been placed is inserted into the test strip receiver on one side of the blood glucose meter. The blood glucose meter tests the blood on the test strip and displays blood glucose data on the screen of the meter, allowing the user to monitor his or her present physiological conditions by reading the blood glucose data displayed on the screen.

However, the existing blood glucose meters not only are expensive but also tend to age after long-term use, thus compromising the accuracy of blood glucose test results. In addition, a blood glucose meter is set with a blood glucose data calibration code which must be changed when a blood glucose test strip of a different brand is used. Therefore, each time a blood glucose meter is used, the user must verify whether the current calibration code corresponds to the brand of the blood glucose test strip being used. As the majority of blood glucose meter users are of advanced ages, such a complicated operating procedure can be troublesome, if not leading to inaccurate blood glucose test results.

Moreover, even though smart phones, laptop computers, tablet computers, and like mobile devices are quite popular nowadays, it has never been seen that the test data generated by a physiological measurement device, such as the aforesaid blood glucose meters, can be directly supplied to a mobile device in order to be read, stored, analyzed, and displayed by the mobile device. As far as blood glucose meters are concerned, this is mainly because they already have a screen for displaying. Nevertheless, as stated above, the existing blood glucose meters are disadvantaged by potential aging after long-term use and an elderly-unfriendly operating procedure, both of which are likely to impair the accuracy of blood glucose test results.

### BRIEF SUMMARY OF THE INVENTION

It is an object of the present invention to provide a blood glucose measuring kit which can display blood glucose data by means of the screen of an external mobile device and whose production cost can therefore be reduced. The blood glucose measuring kit is intended to overcome the foregoing drawbacks of the conventional blood glucose meters, namely the high selling prices, the aging problem resulting from long-term use, and the elderly-unfriendly design of having to verify whether the current calibration code corresponds to the brand of the blood glucose test strip being used, wherein the latter two drawbacks may render blood glucose test results inaccurate.

To achieve the above object, the present invention discloses a blood glucose measuring kit connectable to a mobile device, wherein the measuring kit includes a signal processing program, a housing, a control unit, a test strip receiver, and a data transfer port.

The signal processing program is stored in the Internet and downloaded wirelessly from the Internet to an external mobile device and can be updated wirelessly from the Internet once downloaded to the mobile device.

The control unit, the test strip receiver, and the data transfer port are provided in the housing. Both the test strip receiver and the data transfer port are electrically connected to the control unit.

After an external blood glucose test strip is inserted into the test strip receiver, the control unit generates blood glucose data by testing the blood glucose test strip with the test strip receiver. Then, the control unit sends out a signal indicating completion of the blood glucose test. The data transfer port is electrically connected to the mobile device and transfers the blood glucose data to the mobile device.

The mobile device processes the blood glucose data by means of the signal processing program. More specifically, the mobile device reads, analyzes, applies statistics to, and stores the blood glucose data and displays the blood glucose data on a screen of the mobile device. With the signal processing program, the mobile device can also upload the blood glucose data wirelessly to the Internet and download wirelessly from the Internet the blood glucose data and the calibration code of the blood glucose data.

According to the above, the signal processing program can be downloaded or updated wirelessly from the Internet in advance. To use the blood glucose measuring kit, a blood glucose test strip is inserted into the test strip receiver, which tests the blood glucose test strip in order for the control unit to generate blood glucose data and then send out a blood glucose test completion signal. At the same time, the data transfer port is electrically connected to the mobile device and thus transfers the blood glucose data to the mobile device, where the blood glucose data are processed with the signal processing program. More particularly, the mobile device reads, analyzes, applies statistics to, and stores the blood glucose data and displays the blood glucose data on the screen of the mobile device. The signal processing program also makes it possible to upload the blood glucose data wirelessly from the mobile device to the Internet and to download the blood glucose data and the calibration code thereof wirelessly from the Internet to the mobile device.

In a nutshell, the blood glucose measuring kit of the present invention is configured to test blood glucose test strips but not to display blood glucose data on its own. With the assistance of a mobile device, which is very popular among consumers nowadays, a signal processing program for processing blood glucose data can be downloaded wirelessly from the Internet, so as for the mobile device to read, analyze, apply statistics to, store, and display blood glucose data. Thus, the production cost of the blood glucose measuring kit can be reduced, allowing the blood glucose measuring kit to be replaced at a relatively low price after long-term use, now that the aging of the blood glucose measuring kit may lower the accuracy of blood glucose test results. Besides, the mobile device can automatically download the calibration codes of blood glucose data in a wireless manner, thereby simplifying the operating procedure of blood glucose tests. This makes it easier for an elderly user to correctly operate the blood glucose measuring kit in order to measure blood glucose level, and to correctly operate the mobile device in order to display blood glucose data. Consequently, the object of displaying blood glucose data via the screen of an external mobile device and thereby reducing the production cost of the blood glucose measuring kit is achieved.

Implementation of the present invention is further described as follows.

In addition to the foregoing major structural features, the blood glucose test completion signal may be a light, a sound, or a vibration.

In addition to the foregoing major structural features, the housing is provided with at least one indicator lamp electrically connected to the control unit, and the control unit sends out the blood glucose test completion signal in the form of light by means of the at least one indicator lamp.

In addition to the foregoing major structural features, the housing is provided therein with a data storage unit electrically connected to the control unit. The data storage unit can store blood glucose data and output the stored blood glucose data to the data transfer port.

In addition to the foregoing major structural features, the housing is provided therein with a voice memory unit and a voice amplification and replay unit, both electrically connected to the control unit. The voice amplification and replay unit can read and replay voice-based instruction data stored in the voice memory unit.

In addition to the foregoing major structural features, the housing is provided with an operation interface and a display, both electrically connected to the control unit.

### BRIEF DESCRIPTION OF THE SEVERAL VIEWS OF THE DRAWINGS

The structure as well as a preferred mode of use, further objects, and advantages of the present invention will be best understood by referring to the following detailed description of some illustrative embodiments in conjunction with the accompanying drawings, in which:

FIG. 1 is a perspective view of a preferred embodiment of the present invention;

FIG. 2 is a functional block diagram of the embodiment shown in FIG. 1; and

FIG. 3 is a perspective view showing an additional mode of implementation of the embodiment depicted in FIG. 1.

### DETAILED DESCRIPTION OF THE INVENTION

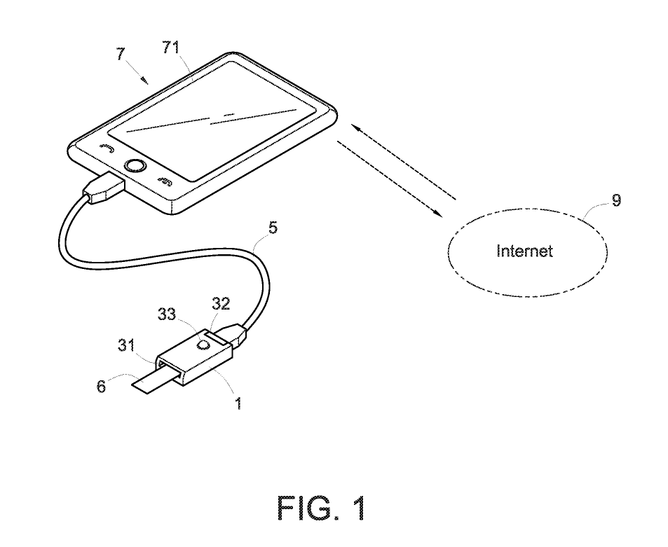

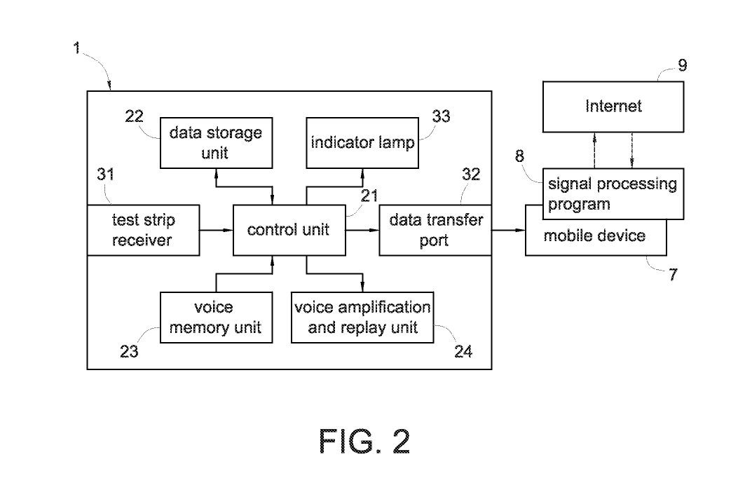

Please refer to FIG. 1 and FIG. 2 for a blood glucose measuring kit according to the present invention which is connectable to a mobile device. The measuring kit includes a signal processing program 8 and a housing 1 provided therein with a control unit 21, a test strip receiver 31, and a data transfer port 32.

The signal processing program 8 is an application software (also known as App) stored in the Internet 9 and having an App ID. With the App ID, the signal processing program 8 can be downloaded wirelessly from the Internet 9 to an external mobile device 7 and, once downloaded, can be updated wirelessly from the Internet 9. The mobile device 7 in this embodiment may be a smart phone, a laptop computer, or a tablet computer.

The test strip receiver 31 is electrically connected to the control unit 21, and so is the data transfer port 32.

The test strip receiver 31 is configured to receive an external blood glucose test strip 6 inserted therein. The control unit 21 can generate blood glucose data by testing the blood on the blood glucose test strip 6 with the test strip receiver 31, and send out a blood glucose test completion signal in the form of light, sound, or vibration. The data transfer port 32 can be electrically connected to the mobile device 7 via a signal transmission cable 5 in order to transfer blood glucose data to the mobile device 7.

The mobile device 7 processes blood glucose data by means of the signal processing program 8. More specifically, the mobile device 7 can read, analyze, apply statistics to, and store blood glucose data and display blood glucose data on a screen 71 of the mobile device 7. Using the signal processing program 8 and the App ID, the mobile device 7 can also upload blood glucose data wirelessly to the Internet 9 and download blood glucose data and the calibration code thereof wirelessly from the Internet 9.

Preferably, the housing 1 is provided with at least one indicator lamp 33 electrically connected to the control unit 21, and the control unit 21 can send out a light via the at least on indicator lamp 33 as the blood glucose test completion signal.

Preferably, the at least one indicator lamp 33 is at least one light-emitting diode (LED), and there may be one or plural indicator lamps 33. The at least one indicator lamp 33 may be so designed that the blood glucose test completion signal is defined by a change in light color.

The present invention can be carried out with the components described above. The signal processing program 8 may be downloaded wirelessly from the Internet 9 to the mobile device 7 or updated wirelessly from the Internet 9 in advance. The data transfer port 32 may be electrically connected to the mobile device 7 via the signal transmission cable 5 before the blood glucose measuring kit is used.

To use the measuring kit, a blood glucose test strip 6 is inserted into the test strip receiver 31, so as for the test strip receiver 31 to test the blood on the blood glucose test strip 6 and for the control unit 21 to generate blood glucose data accordingly. Then, the control unit 21 sends out a blood glucose test completion signal to notify the user that the blood glucose test strip 6 has been tested.

With the signal processing program 8, the mobile device 7 reads the blood glucose data through the data transfer port automatically, and the blood glucose data are thus automatically transferred to the mobile device 7.

The signal processing program 8 processes the blood glucose data automatically. More specifically, the signal processing program 8 enables the mobile device 7 to read, analyze, apply statistics to, and store the blood glucose data and display the blood glucose data on the screen 71 of the mobile device 7.

Then, using the signal processing program 8 and the App ID, the mobile device 7 not only can automatically and wirelessly upload the blood glucose data to the Internet 9, but also can automatically and wirelessly download from the Internet 9 any previously uploaded blood glucose data and the blood glucose data calibration code corresponding to the blood glucose test strip 6 being used.

Thus, the blood glucose measuring kit of the present invention is capable of testing blood glucose test strips 6 but not of displaying blood glucose data on its own. The blood glucose measuring kit is designed to work in conjunction with the mobile device 7, which is a very popular product on the market and to which the signal processing program 8 for processing blood glucose data can be downloaded automatically and wirelessly from the Internet 9 using the App ID, so as for the mobile device 7 to read, analyze, apply statistics to, store, and display blood glucose data automatically. As the housing 1 of the blood glucose measuring kit houses only the test strip receiver 31, the control unit 21, the data transfer port 32, and the at least one indicator lamp 33, the measuring kit is structurally simple and can be significantly downsized to lower its production cost. In fact, the production cost can be so low that the measuring kit may be given free of charge to those who buy blood glucose test strips 6. The small volume of the measuring kit also provides increased portability.

Due to its low selling price, the blood glucose measuring kit can be replaced whenever necessary, especially when it ages after long-term use, thereby preventing the accuracy of blood glucose test results from being compromised. In addition, the ability of the mobile device 7 to automatically and wirelessly download blood glucose data calibration codes greatly simplifies the operating procedure of blood glucose tests, allowing an elderly user to correctly operate the blood glucose measuring kit and the mobile device 7 in measuring blood glucose level and displaying blood glucose data respectively. Thus, the object of displaying blood glucose data via the screen 71 of the external mobile device 7 and reducing the production cost of the blood glucose measuring kit is attained. The aforementioned problems of the prior art, i.e., the high selling prices of the conventional blood glucose meters, the aging of the conventional blood glucose meters after long-term use, and the elderly-unfriendly operating procedure required to make sure that the current calibration code corresponds to the brand of the blood glucose test strip being used, are also solved, wherein the latter two problems may contribute to inaccurate blood glucose test results. Furthermore, the blood glucose measuring kit and the mobile device 7 can be used with ease.

The present invention may further include the following technical features:

The housing 1 is preferably provided therein with a data storage unit 22 electrically connected to the control unit 21. The data storage unit 22 can store the blood glucose data received from the control unit 21 and output the stored blood glucose data to the data transfer port 32 through the control unit 21.

The blood glucose measuring kit of the present invention can be used in such a way that the test strip receiver 31 completes the testing of a blood glucose test strip 6 in an offline state, i.e., before the data transfer port 32 is electrically connected to the mobile device 7. In that case, the blood glucose data are stored in the data storage unit 22 and will be automatically transferred to the mobile device 7 as soon as the data transfer port 32 is electrically connected to the mobile device 7.

Therefore, even if the mobile device 7 is not carried along with the blood glucose measuring kit, blood glucose level can still be tested with the blood glucose measuring kit whenever desired, for the resulting multiple entries of blood glucose data can accumulate in the data storage unit 22 and be transferred to the mobile device 7 in one batch.

Preferably, the housing 1 is further provided therein with a voice memory unit 23 and a voice amplification and replay unit 24, both electrically connected to the control unit 21, wherein the voice amplification and replay unit 24 is configured to read and replay voice-based instruction data pre-stored in the voice memory unit 23.

Thus, when a blood glucose test strip 6 is inserted into the test strip receiver 31, the control unit 21 can drive the voice amplification and replay unit 24 to replay the voice instruction, thereby instructing the user to operate the blood glucose measuring kit properly.

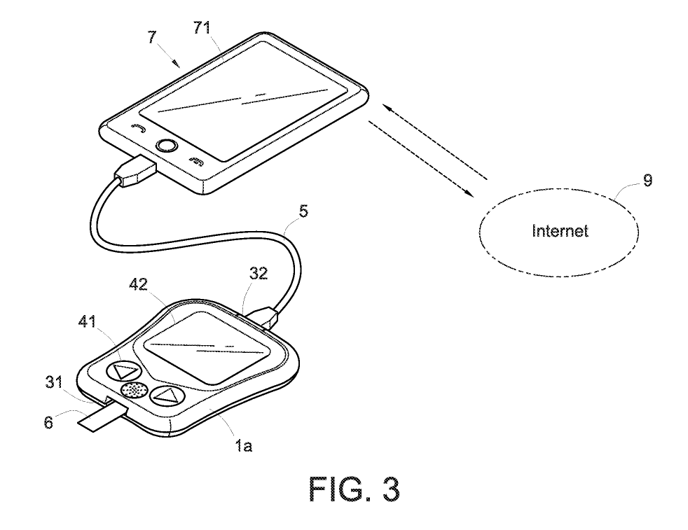

FIG. 3 is a perspective view of an additional mode of implementation of the embodiment shown in FIG. 1. As shown in FIG. 3, the housing la is further provided with an operation interface 41 and a display 42, both electrically connected to the control unit 21, and the at least one indicator lamp 33 is dispensed with.

In this embodiment, blood glucose tests can be performed by operating the operation interface 41 on the housing la, and blood glucose data are directly displayed on the display 42 on the housing la. The rest of the components and the method of implementation are the same as those in the previous embodiment.

What is claimed is:

1. A blood glucose measuring kit connectable to a mobile device, comprising:
a signal processing program stored in the Internet and downloaded wirelessly from the Internet to an external mobile device, wherein the signal processing program, once downloaded to the mobile device, can be updated wirelessly from the Internet; and
a housing provided therein with a control unit, a test strip receiver electrically connected to the control unit, and a data transfer port electrically connected to the control unit;
wherein after an external blood glucose test strip is inserted into the test strip receiver, the control unit generates blood glucose data by testing the blood glucose test strip with the test strip receiver and sends out a blood glucose test completion signal, and the data transfer port, once electrically connected to the mobile device, transfers the blood glucose data to the mobile device, so as for the mobile device to process the blood glucose data through the signal processing program by reading, analyzing, applying statistics to, and storing the blood glucose data and displaying the blood glucose data on a screen of the mobile device, the signal processing program also enabling the mobile device to upload the blood glucose data to the Internet wirelessly and to download wirelessly from the Internet the blood glucose data and a calibration code of the blood glucose data.

2. The blood glucose measuring kit of claim 1, wherein the blood glucose test completion signal is a light, a sound, or a vibration.

3. The blood glucose measuring kit of claim 1, wherein the housing is provided with at least one indicator lamp electrically connected to the control unit, and the control unit sends out a light as the blood glucose test completion signal through the at least one indictor lamp.

4. The blood glucose measuring kit of claim 1, wherein the housing is provided therein with a data storage unit electrically connected to the control unit, the data storage unit being configured for storing the blood glucose data and outputting the blood glucose data to the data transfer port.

5. The blood glucose measuring kit of claim 1, wherein the housing is provided therein with a voice memory unit and a voice amplification and replay unit, both electrically connected to the control unit, the voice amplification and replay unit being configured for reading and replaying voice-based instruction data stored in the voice memory unit.

6. The blood glucose measuring kit of claim 5, wherein the housing is provided with an operation interface and a display, both electrically connected to the control unit.
